(19) United States
(12) Patent Application Publication
(10) Pub. No.: US 2005/0175635 A1
(43) Pub. Date: Aug. 11, 2005

(54) Method and composition

(76) Inventor: Camilo Anthony Leo Selwyn Colaco, Cambridge (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/204,829

(22) PCT Filed: Feb. 22, 2001
(86) PCT No.: PCT/GB01/00752

(30) Foreign Application Priority Data
Feb. 23, 2000 (GB) 0004547.6

(51) Int. Cl. A61K 39/38

(52) U.S. Cl. 424/234.1

(57) ABSTRACT

The present invention relates to a method for isolating and identifying specific immunogenic pathogen-specific peptides associated with stress proteins induced by the treatment of pathogens and pathogen-infected cells with stress inducing stimuli. The invention also relates to the use of the antigenic fragments derived from complexes thereof with heat shock or other stresses proteins as the immunogenic determinant in vaccine compositions.

The present invention relates to a method for making a vaccine composition and to vaccine compositions containing isolated antigenic fragments. The invention also relates to a method for identifying candidate vaccine antigens associated with stress-induced proteins so that those antigen fragments can be made by this or other techniques for incorporation into vaccine compositions.

### BACKGROUND OF THE INVENTION

An important component of any human immune response to an infection or illness is the presentation of antigens to T cells by antigen presenting cells (APCs) such as macrophages, B cells or dendritic cells. Fragments of foreign antigens, hereinafter called antigenic fragments, are presented on the surface of the macrophage in combination with Major Histocompatibility Complex (MHC) molecules, in association with helper molecules, such as CD4 and CDB molecules. Such antigenic fragments presented in this way are recognised by the T cell receptor (TCR) of T cells, and the interaction of the antigen with the TCR results in antigen-specific T cell proliferation, and secretion of specific lymphokines by the T-cells. The nature of the antigenic fragment presented by the APCs is critical in establishing immunity in a mammal.

Heat shock proteins (HSPs) form a family of highly conserved proteins that are widely distributed throughout the plant and animal kingdoms. On the basis of their molecular weights, HSPs are grouped into six different SUBSTITUTE SHEET (RULE 26) families: small (hsp 20-30 kDa); hsp40; hsp60; hsp70; hsp90 and hsp100. Although HSPs were originally identified in cells subjected to heat stress, they have been found to be associated with many other forms of stress, such as infections, and are thus more commonly known as “stress proteins” (SPs).

Members of the mammalian hsp90 family include cytosolic hsp90 (hsp83) and the endoplasmic reticulum counterparts hsp90 (hsp83), hsp87, Grp94 (Erp99) and gp97, see for instance, Gething et al. (1992) Nature 355: 33-45. Members of the hsp70 family include cytosolic hsp70 (p73) and hsp70 (p72), the endoplasmic reticulum counterpart BiP (Grp78), and the mitochondrial counterpart hsp70 (Grp75). Members of the mammalian hsp60 family have only been identified in the mitochondria. The latter family of HSPs is also found in parasitic organisms such as helminths and protozoa, as well as procaryotes which also contain three other major families of HSPs, the GroEL, GroES, DnaJ and DnaK families. As in eucaryotes, the procaryotic HSPs are also thought to function in the folding of nascent polypeptide chains during protein synthesis.

In eucaryotic cells which have intracellular membrane organelles, one of the roles of HSPs is to chaperone antigenic fragment peptides from one cellular compartment to another and to present those peptides to the MHC molecules for cell surface presentation to the immune system. In the case of diseased cells, HSPs also chaperone viral or tumour-associated peptides to the cell-surface, see Li and Sirivastava (1994) Behring Inst. Mitt, 94: 37-47 and Suzue et al. (1997) Proc. Natl. Acad. Sci. USA 94: 13146-51. The chaperone function is accomplished through the formation of complexes between HSPs and proteins and between HSPs and viral or tumour-associated peptides in an ATP-dependent reaction. HSPs bind a wide spectrum of peptides in an ATP-dependent manner. The bound peptides appear to be a random mix of peptides. The mixtures and exact natures of the peptides have not been determined. The association of HSPs with various peptides has been observed in normal tissues as well and is not a tumour-specific phenomenon, see Srivastava (1994) Experimentia 50: 1054-60.

In a therapeutic context, it has been proposed to use mammalian HSPs as vaccines, see for example WO 97/10000 and WO 97/10001. These disclose that a mixture of HSPs isolated from cancer cells or virally infected cells are capable of eliciting protective immunity or cytotoxic T lymphocytes to the cognate tumour or viral antigen. However, in contrast, HSPs isolated from normal cells are unable to elicit such immunity. It is now thought that HSPs are not immunogenic per se, but are able to elicit immunity because of their association with tumour or virus specific antigenic fragment peptides that are generated during antigen processing. Specifically, the peptides associated with the HSPs are immunogenic, and are presented to the T cells. HSPs stripped of associated peptides lose their immunogenicity (Udono, H. and Srivastava, P. K., Journal of Experimental Medicine, 178, page 1391 ff, 1993). To date, the nature of these peptides has not been determined.

The use of mammalian HSP/antigenic fragment complexes from infected cells as vaccines against intracellular pathogens has been disclosed in WO 95/24923. HSPs isolated from viral infected cells have been suggested as a source of antigenic peptides, which could then be presented to T cells. This necessitates the production and purification of HSPs from such cells. The use of HSP proteins as vaccine components has further been disclosed in WO 97/10000, WO 97/10001 and WO 97/100002 which disclose that a mixture of heat shock proteins (HSPs) isolated from cancer cells or virally infected cells are capable of eliciting protective immunity or cytotoxic T lymphocytes to the cognate tumour or viral antigen. HSPs isolated from tumour cells or viral infected cells have been suggested as a source of antigenic peptides, which could then be presented to T cells. Furthermore WO 98/34641 discloses that surprisingly low amounts of HSPs are required to immunise animals against tumour or viral antigens. All these HSP vaccine approaches utilise mammalian HSPs from the species for immunisation of the desired animal species.

HSPs from the extracellular pathogens themselves have also been utilised to immunise mammalian species as antigens per se but not as carriers of antigenic peptides except as conjugates or hybrid fusion proteins. Thus WO 95/14093 discloses that the use of Helicobacter pylori HspA and B as immunogens elicits a good antibody response against these proteins that is effective against the organism. Similarly, WO 96/40928 discloses that the use of HSP 70 and 72 from Streptococcus elicits a good antibody response against these proteins that is effective against the organism. Furthermore, WO 90/02564 discloses that the use of Trypanosomal, Mycoplasmal or Mycobacterial HSPs, and especially HSP70, as immunogens elicits a good antibody response against these proteins that should be effective against the respective organisms.

Alternatively U.S. Pat. No. 05,830,475 uses proteins expressed as fusions of the M. Bovis HSP genes as antigens and U.S. Pat. No. 05,736,164 uses the T-cell epitope of hsp65 conjugated to poorly immunogenic antigens.

In the above proposals, the antigenic fragments have been used in the form of complexes with the heat shock or other stress proteins, which were considered necessary to provide the chaperone function described above.

Surprisingly, we have found that it is not necessary to use the stress-protein/antigenic fragment complexes described above in the vaccine composition, but that the antigenic fragment bound to the HSP or other stress protein is effective as a vaccine without the associated HSP or other stress protein hitherto considered as a necessary carrier or chaperone for the antigenic fragment. The stress-protein/antigenic fragment complexes used hitherto and particularly SPs induced by a stress-inducing stimuli, carry the active antigenic fragment and this can readily be isolated from the complex and used as a vaccine composition per se. Furthermore, the nature of the isolated antigenic fragment can readily be identified and this then enables the proteins making up the antigenic fragment to be prepared by a number of other conventional synthesis techniques without the need to use stress protein complexes. Since the initial stress protein technique has preferentially selected the active antigenic fragment to be bound to the stress protein to treat the specific infection or illness, the problem of identifying which antigenic fragment to synthesise has been overcome. The invention thus also provides a method for identifying the nature of the active antigenic fragment to be synthesised for inclusion in a vaccine composition for the treatment of a specific illness or infection or a range of illnesses or infections, which method comprises isolating the antigen fragment bound to a stress protein complex and identifying that fragment, for example by DNA or other techniques, to identify the nature of the immunogenic agent to be included in the vaccine composition.

### SUMMARY OF THE INVENTION

Therefore, in a first aspect, the present invention provides a method for identifying candidate vaccine antigenic fragments, which method comprises the steps of:
a) exposing pathogenic organisms or cells infected thereby to stress-inducing stimuli;
b) extracting the endogenous stress induced proteins from the treated organisms or cells; and
c) identifying the antigenic fragments associated with the extracted stress induced proteins.

The invention further provides a method for making a vaccine composition, which method comprises the use as the immunogenic component of the composition of at least one of the antigenic fragments extracted from a stress induced protein produced by exposing a pathogenic organism or a cell infected by a pathogenic organism to a stress inducing stimulus.

The invention further provides a method for making a vaccine composition, which method comprises identifying the requisite immunogenic antigenic fragment associated with a stress induced protein; synthesising that antigenic fragment and using that synthesised fragment as the immunogenic component of a vaccine composition. In a further embodiment the invention the synthesised immunogenic antigenic fragment may be contained within a larger parent molecule.

In a further embodiment the invention also provides the use in the preparation of a vaccine composition for the treatment of an illness or infection caused by a pathogenic organism of a synthetic immunogenic peptide or mixture of peptides, which has been selected according to the identification of the antigenic fragment peptides in a complex thereof with a stress induced protein produced by exposing that pathogenic organism or a cell infected by that pathogenic organism to a stress inducing stimulus.

The invention also provides a vaccine composition for administration to a mammal so as to elicit an immune response from that mammal, said composition comprising an immunogenic determinant, characterised in that the immunogenic determinant comprises one or more antigenic fragments derived from or identified from a complex of the antigenic fragment with a stress protein, which complex has been produced by exposing a pathogenic organism or a cell infected by that pathogenic organism to a stress inducing stimulus.

The term “vaccine” as used herein, refers to any composition which stimulates the immune system such that it can better respond to subsequent illness or infections. It will be appreciated that a vaccine usually contains an immunogenic determinant and an adjuvant, which non-specifically enhances the response to that determinant.

The term “pathogenic organism” as used herein, refers to any pathogen that causes a disease or illness in a vertebrate, including bacterial, protozoan and fungal species as well multi-cellular parasitic pathogens.

The term “stress proteins” and “heat shock protein”, as used herein, are standard in the art, and includes those proteins that comprise the GroEL, GroES and DnaK and DnaJ families of bacterial HSPs and related families in other extra-cellular pathogens.

The stress-inducing stimuli used to induce the formation of stress induced protein/antigenic fragment complexes for present use include environmental stimuli, for example treatment with heat or with a salt, compound or complex; or cellular stimuli, for example treatment with inflammatory monokines or cytokines. The stress stimuli to which the pathogen is exposed may be applied by any suitable in vitro technique used in the immunobiology art, for example cultivation under limited nutrient levels, or osmotic shock of a pathogen once it has been cultivated to stationary growth by the addition of high concentrations of an electrolyte such as NaCl to the cultivation medium. We prefer to apply the stress by a heat treatment of the pathogen at a temperature 5-8° C. above the normal growth temperature of the organism. Typically, the pathogen will be cultivated under conventional growth conditions to the stationary state. Samples of the active pathogen culture can then be taken and cultivated again but the temperature of cultivation is increased during the second cultivation stage to the elevated temperature required to induce production of the stress proteins. Without being constrained by theory, it is thought that the treatment operates either to induce specifically those stress proteins most able to interact with antigenic peptides, or to induce those stress proteins which are most easily phagocytosed by APCs, or both. The optimum conditions for inducing the stress proteins can readily be determined by simple trial and error and the effect of a change of stimuli assessed using conventional techniques, such as in vivo testing on animals or by other techniques, for example those described in ‘Current Protocols in Immunology’, Wiley Interscience, 1997.

It is particularly preferred that where the organism is an extra-cellular procaryotic agent or a parasitic agent, including multi-cellular and protozoan species, the stress proteins extracted are the stress protein complexes obtained by treatment of these organisms by heat shock or other enviromental stresses or, for those pathogens that carry receptors for them, cytokine treatment. For intra-cellular procaryotic and parasitic agents, including multi-cellular and protozoan species, the stress proteins extracted are those stress protein complexes obtained from cells infected by these organisms and treated with cytokines to induce synthesis of stress-protein complexes. Preferably, the cytokine interferon, and more preferably interferon alpha, is used for the induction of the appropriate set of stress proteins in virally infected cells which are then extracted to identify candidate viral vaccine antigens. Preferably, tumour necrosis factor, and more preferably tumour necrosis factor alpha, is used as the cytokine for the induction of the appropriate set of stress proteins in parasite infected cells which are then extracted to identify candidate parasite vaccine antigens. Alternatively, the cytokine tumour necrosis factor, and more preferably tumour necrosis factor alpha, may be used for the induction of the appropriate set of stress proteins directly in the parasitic pathogens that contain tumour necrosis factor receptors, and these stress protein complexes are then extracted to identify candidate parasite vaccine antigens. For identification of candidate bacterial and protozoan candidate vaccine antigens, the stress-protein complexes extracted are those induced by heat or heavy-metal stress in the bacterial and protozoan pathogens.

We have described in our co-pending PCT Applications Nos GB99/02607, 00/03225 and 00/03228 suitable in vitro methods for the production of the stress protein/antigenic fragment complexes and those are suitable for use in the present invention.

The extraction and purification of the stress protein/antigenic fragment complexes for use in the present invention from the extra-cellular pathogen is standard in the art. Suitable methods include disruption of treated organism by homogenisation or sonication, followed by centrifugation to obtain a crude complex preparation in the supernatant. Optionally, the complexes may be purified further by the use of ADP binding columns or other suitable methods readily available to the person skilled in the art, see for example those described in WO 97/10000 and WO 97/10001.

The antigenic fragment peptides can be isolated from the stress protein complex containing them by any suitable technique. Preferably, the antigenic fragment peptides are eluted from the stress protein complexes by a wide range of conventional extraction techniques, for example treatment of the complexes with adenosine triphosphate. Alternatively the associated peptides are eluted from the extracted stress-protein complexes by their treatment with chaotropic agents, for example guanidine hydrochloride and ionic detergents and/or denaturation of the SP vehicle.

As stated above, the antigenic fragments isolated from the stress protein complexes can be used as the immunogenic determinant in a vaccine composition as described below. Alternatively, since the antigenic fragments contain those peptides which are effective against the illness or infection caused by the pathogen from which the stress protein complexes have been prepared, the preparation of the complexes provides a mechanism for selecting the desired peptides from amongst many possible peptides which it would be desirable to synthesise for the preparation of a vaccine composition which is effective against that illness or infection. The nature of the peptides in the isolated antigenic fragment peptides may be identified by methods well known in the art, including N-terminal sequencing using a gas-phase sequencer or by mass spectroscopic analysis. Partial amino acid sequence can then be used to identify corresponding vaccine candidates from sequence database searches or cloning of identified antigens using recombinant DNA techniques.

Having isolated and identified the desirable peptides for use in the treatment of a specific illness or infection, that peptide or mixture of peptides can readily be synthesised using any suitable technique, for example recombinant DNA techniques, to provide a synthetic antigenic fragment for use in the preparation of a vaccine. Alternatively, the synthetic antigenic fragment may be contained within a larger molecule, including the parent molecule from which it may have originally derived.

The isolated peptides or ones synthesised from a knowledge of the identification of the antigenic fragments in the stress protein complex may be used singly or in various combinations to yield compositions suitable for use as prophylactic and therapeutic vaccines for infectious diseases. Such a vaccine may be given by any suitable means, such as orally, or by injection.

Preferably the vaccine composition contains at least one antigenic fragment from or identified from the stress protein complex derived from the stressed pathogen. We particularly prefer that the GroEL, GroES, DnaK and DnaJ families of proteins are used as immunogenic determinants in the present invention, with DnaJ and GroEL most preferred. Comparative immunogenicity can be determined by in vivo testing on animal models. Other suitable methods will be readily apparent to the person skilled in the art, see ‘Current Protocols in Immunology’, Wiley Interscience, 1997.

Thus, from another aspect, the present invention provides a method for eliciting an immune response from an animal to infection by a pathogenic organism which comprises administering a vaccine containing an immunogenic determinant, characterised in that the immunogenic determinant is an antigenic peptide fragment isolated from or identified from the complex produced as described above.

The antigenic peptide fragment of the vaccine of the present invention may be delivered in combination with an adjuvant and in an aqueous carrier. Suitable adjuvants are readily apparent to the person skilled in the art, and include for example Freund's complete adjuvant, Freund's incomplete adjuvant, Quil A, Detox, ISCOMs or squalene. However, the vaccine compositions of the present invention may also be effective without an adjuvant. However, it will be appreciated that the vaccine of the present invention may also be effective with the use of stress proteins themselves as adjuvants.

The invention also provides a method for treating an animal with a vaccine of the invention by administering a pharmaceutically acceptable quantity of the vaccine of the invention, optionally in combination with an adjuvant, sufficient to elicit an immune response in the animal.

The animal is typically a human. However, the invention can also be applied to the treatment of other mammals such as horses, cattle, goats, sheep or swine, and to the treatment of birds, notably poultry such as chicken or turkeys.

The vaccine compositions of the present invention may be administered by any suitable means, such as orally, by inhalation, transdermally or by injection and in any suitable carrier medium. However, it is preferred to administer the vaccine as an aqueous composition by injection using any suitable needle or needle-less technique.

The vaccines of the invention may contain any suitable concentration of the antigenic peptide fragments. We prefer that the antigenic fragment is administered in the range of 10-600 μg, preferably 10-100 μg, most preferably 25 μg, per Kg of body weight of the animal being treated. It will be appreciated that the vaccine of the invention may be applied as an initial treatment followed by one or more subsequent treatments at the same or a different dosage rate at an interval of from 1 to 26 weeks between each treatment to provide prolonged immunisation against the pathogen.

The following examples are provided to illustrate but not limit the invention and all parts and percentages are given by weight.

### EXAMPLE 1

M. Bovis was grown to stationary phase and heat-shocked at 42° C. for 0.5 hr or at 39° C. for 5 hr and cultured overnight. Cells were then washed in phosphate buffered saline (PBS) and re-suspended in 10 mM phosphate pH 7.4 with 2 mM MgCl₂ as a hypotonic homogenisation buffer. The cells were then disrupted using a Waring blender cell homogeniser. Alternatively, the cells can be lysed by the use of detergents such as Tween or Triton, complement lysis at 37° C. or by repeated freeze-thaw cycles (e.g. in liquid nitrogen). The cell lysate is then treated by centrifugation, typically 3-5000×g for 5 minutes, to remove the nuclear and cell debris, followed by a high speed centrifugation step, typically 100,000 g for 15-30 minutes.

The supernatant thus obtained may be purified by ammonium sulphate precipitation which uses a 20-70% ammonium sulphate cut. Specifically, 20% (w/w) ammonium sulphate is added at 4° C., the precipitate is discarded, followed by the addition of more ammonium sulphate to bring the concentration to 70%.

The protein precipitate is harvested by centrifugation, and then dialysed into saline as a physiological, injectable buffer, to remove the ammonium sulphate before use. The stress protein/antigenic fragment complexes may be further purified from the supernatant by affinity chromatography on matrices carrying adenosine diphosphate, such as ADP-agarose or ADP-sepharose as described in WO 97/10000, WO 97/10001 and WO 97/10002.

Pathogen-specific peptides associated with the isolated stress protein complexes are then released from the complex by treatment with 0.1-10 mM adenosine triphosphate, separated and further purified by chromatography on reverse phase columns or capillary-zone electrophoresis. The isolated peptides are sequenced to identify them by N-terminal sequencing using a gas-phase sequenator or mass spectroscopy including MALDI-TOFT. The parent proteins from which the identified peptide sequences were derived could be determined by searching available DNA or protein sequence databases and these used to yield vaccines.

### EXAMPLE 2

Immunogenicity of the isolated and identified peptides produced according to Example 1 may be determined by in vivo immunisation studies or in vitro T cell proliferation assays. Suitable assays include the mixed-lymphocyte reaction (MLR), assayed by tritiated thymidine uptake, and cytotoxicity assays to determine the release of ⁵¹Cr from target cells, see ‘Current Protocols in Immunology’, Wiley Interscience, 1997). Alternatively, antibody production may be examined, using standard immunoassays or plaque-lysis assays, or assessed by interuterine protection of a foetus, see ‘Current Protocols in Immunology’. Suitably immunogenic peptides may be used either singly or in combination to yield vaccines against infectious diseases.

A mixture of antigenic fragment peptides were prepared as described in Example 1 above and mice and rabbits were vaccinated with 1-100 micrograms of the mix complexed with alum in phosphate buffered saline. The initial immunisation was boosted with identical vaccine dosages a month after the primary injection. Induction of immunity to pathogen was assayed by Western blot analysis using total M. bovis proteins. Antibody titres against M. Bovis were routinely obtained and cytotoxic T-cell activity directed against pathogen infected cells could also be detected in the immunised mice.

### EXAMPLE 3

Mycobacterium Tuberculosis was grown to saturation for 3 days at 37° C. in Sauton's medium. 4 ml aliquots of the stationary cultures were used to inoculate 500 ml of Sauton's medium in a 2 litre conical flask and the cultures grown overnight at 30° C. The log phase cultures were then raised to 40° C. and grown for a further 4 hrs before the bacteria were harvested by centrifugation at 10,000 rpm for 10 minutes. Non-induced (constitutive) shock proteins were isolated by centrifugation from the initial cell cultures at 37° C.

Cell pellets from the centrifuged samples were re-suspended in lysis solution containing 0.5% Tween and the stress protein/antigenic fragment complexes were prepared from induced and non-induced cells using ammonium sulphate precipitation as in Example 1 above. The purified stress protein complexes were re-suspended in 10% acetic acid and boiled for 15 mins to elute the associated antigenic fragment peptides from the complexes. The denatured HSPs were pelleted in a Beckman airfuge for 30 mins in a cold room and the peptide-containing supernatants harvested by freeze-drying.

Immunisation of mice with the peptide fragments isolated from the complexes was achieved by administration of the peptide fragments with a variety of adjuvants including Quil and ISCOMs and gave significant protective immunity to live challenge with M. Tuberculosis, as assessed by lung colony counts.

### EXAMPLE 4

Cell lines infected with the malarial pathogen plasmodium were incubated in a serum free media, such as RPMI (Sigma), and incubated with tumour necrosis factor alpha (TNF-a) overnight. Infected cell cultures were grown overnight in the presence or absence of lug/ml TNF-α at 37° C., for the isolation of constitutive or TNF-induced SPs, or heat-shocked by incubation at 42° C. for 2 hrs for the isolation of heat-induced stress proteins (HSPs). Treated cells were pelleted by centrifugation at 3000 g for 5 minutes and re-suspended in a lysis solution of 1% Tween in 100 mM Tris-HCl, pH8. The cell lysate was centrifuged at 5000 g for 5 minutes to remove the nuclei and cell debris, followed by a high speed centrifugation step at 100,000 g for 15-30 minutes. The stress protein/antigenic fragment complexes were prepared from the cleared lysates by ammonium sulphate precipitation as described in Example 1 above.

Associated peptides were eluted from the purified complexes by re-suspending the precipitated complexes in 10% acetic acid and boiling for 15 minutes to dissociate the complexes. The denatured stress proteins were pelleted in a Beckman airfuge for 30 mins in a cold room and the peptide containing supernatants harvested by freeze-drying.

Immunisation of mice with the peptide fragments isolated from the complexes administered with a variety of adjuvants including Quil and ISCOMs gave significant protective immunity as assessed by antibody production.

1. A method for identifying candidate vaccine antigenic fragments to provide an immune response against a pathogenic organism, comprising the steps of:
a) exposing the pathogenic organisms or cells infected thereby to one or more stress-inducing stimuli;
b) extracting the endogenous stress induced proteins from the treated organisms or cells; and
c) identifying the antigenic fragments derived from the pathogenic organism and associated with the extracted stress induced proteins.

2. The method as claimed in claim 1, wherein an antigenic peptide is subsequently synthesised to correspond substantially to the identity of the antigenic fragment derived from the pathogenic organism and associated with the extracted stress induced protein.

3. The method as claimed in claim 2, wherein the antigenic peptide is synthesised using recombinant DNA techniques.

4. A method for making a vaccine composition, which method comprises combining with a pharmaceutically acceptable carrier at least one of the antigenic peptide fragments obtained from a stress induced protein produced by exposing a pathogenic organism or a cell infected by a pathogenic organism to a stress inducing stimulus.

5. A method for making a vaccine composition, which method comprises identifying an immunogenic antigenic fragment associated with a stress induced protein; synthesising that antigenic fragment and combining that synthesised fragment with a pharmaceutically acceptable carrier to form said vaccine composition.

6. A method for the treatment of an individual for an illness or infection caused by a pathogenic organism, comprising administering to said individual an effective amount of a vaccine composition comprising antigenic fragments identified according to claim 1, or synthetic analogues thereof, wherein said antigenic fragments are derived from the pathogenic organism and are not complexed to stress proteins contained in the vaccine.

7. A method for eliciting an immune response from an animal to infection by a pathogenic organism which comprises administering a vaccine containing an immunogenic determinant, wherein the immunogenic determinant is an antigenic peptide fragment isolated from or identified from a complex with a stress induced protein produced by exposing that pathogenic organism or a cell infected by that pathogenic organism to a stress inducing stimulus.

8. A vaccine composition for administration to a mammal so as to elicit an immune response from that mammal, said composition comprising an immunogenic determinant, wherein the immunogenic determinant comprises one or more antigenic fragments identifiable according to claim 1 or synthetic analogues thereof.

9. A method for treating an animal with a vaccine composition as claimed in claim 8, wherein the method comprises administering a pharmaceutically acceptable quantity of the vaccine sufficient to elicit an immune response in the animal.

10. The method as claimed in claim 1, wherein the stress-inducing stimulus is heat.

11. The method as claimed in claim 1, wherein the antigenic fragment contains or more of the GroEL, GroES, DnaK or DnaJ proteins.

12. The method as claimed in claim 1, wherein the antigenic fragment is isolated from the complex with the stress protein by treatment of the complex with adenosine triphosphate or a chaotropic agent.

13. The method as claimed in claim 1, wherein peptides in the antigenic fragment are identified by N-terminal sequencing.

14. The method as claimed in claim 1, wherein the stress inducing stimulus is an environmental stress.

15. The method as claimed in claim 14, wherein the environmental stress stimuli is heat.

16. The method as claimed in claim 1 wherein the stress-inducing stimulus is a heavy-metal treatment.

17. The method as claimed in claim 1, wherein the stress-inducing stimulus is cytotoxic-drug treatment.

18. The method as claimed in claim 1, wherein the stress-inducing stimulus is a free-radical treatment.

19. The method as claimed in claim 1 wherein the stress inducing stimuli is a cellular stress.

20. The method as claimed in claim 1, wherein the stress-inducing stimulus is cytokine treatment.

21. The method as claimed in claim 20, wherein the cytokine is interferon.

22. The method as claimed in claim 20, wherein the cytokine is tumour necrosis factor.

24. The method as claimed in claim 4, wherein the antigenic fragment contains or more of the GroEL, GroES, DnaK or DnaJ proteins.

25. The method as claimed in claim 4, wherein the antigenic fragment is isolated from the complex with the stress protein by treatment of the complex with adenosine triphosphate or a chaotropic agent.

26. The method as claimed in claim 4, wherein peptides in the antigenic fragment are identified by N-terminal sequencing.

27. The method as claimed in claim 4, wherein the stress inducing stimuli is selected from the group consisting of environmental stress, heat, heavy metal treatment, cytotoxic drug treatment, free-radical treatment, and cytokine treatment.

28. The method as claimed in claim 5, wherein the antigenic fragment contains or more of the GroEL, GroES, DnaK or DnaJ proteins.

29. The method as claimed in claim 5, wherein the antigenic fragment is isolated from the complex with the stress protein by treatment of the complex with adenosine triphosphate or a chaotropic agent.

30. The method as claimed in claim 5, wherein peptides in the antigenic fragment are identified by N-terminal sequencing.

31. The method as claimed in claim 1, wherein the antigenic fragment contains or more of the GroEL, GroES, DnaK or DnaJ proteins.

32. The method as claimed in claim 1, wherein peptides in the antigenic fragment are identified by N-terminal sequencing.
